(19) United States
(12) Patent Application Publication
(10) Pub. No.: US 2015/0307829 A1
(43) Pub. Date: Oct. 29, 2015

(54) MOBILE FACILITY FOR PREPARING AND DISTRIBUTING CELL-BASED MEDICINAL PRODUCTS

(71) Applicant: Promethera Biosciences, Mont-Saint-Guibert (BE)

(72) Inventors: Claude Dedry, Mont-Saint-Guibert (BE); Philippe Ducarme, Mont-Saint-Guibert (BE); Eric Halioua, Mont-Saint-Guibert (BE)

(21) Appl. No.: 14/670,019

(22) Filed: Mar. 26, 2015

Related U.S. Application Data
(63) Continuation of application No. PCT/EP2013/070276, filed on Sep. 27, 2013.

(30) Foreign Application Priority Data
Sep. 28, 2012 (EP) 12186677.6

(51) Int. Cl. C12M 1/00; B01L 1/00; C12M 1/02; B62D 65/02; C12M 3/00

(52) U.S. Cl. CPC C12M 23/52; B62D 65/02; C12M 23/44; C12M 41/18; B01L 1/00

(57) ABSTRACT

Example methods, apparatus and system are disclosed relating to a facility for formulating Cell-Based Medicinal Products (CBMPs) in a site where the facility is moved and CBMP formulations are administered. An example facility includes a unitary structure that is assembled together with a vehicle and that houses the working areas and equipment allowing operators to store, count, test, sample, formulate, and/or package CBMPs according to Good Manufacturing Practices. The teachings of the present disclosure relate to a method of providing formulations of a Cell-Based Medicinal Product in sites where the formulations are administered to patients, and from where the facility may be quickly relocated.

### RELATED APPLICATIONS

This patent arises from a continuation of International Patent Application Serial No. PCT/EP2013/070276, filed Sep. 27, 2013, which claims priority to European Patent Application 12186677.6, filed on Sep. 28, 2012, both of which are hereby incorporated herein by reference in their entireties.

### TECHNICAL FIELD

The present disclosure relates to the preparation and the distribution of cell-based medicinal products to medical facilities.

### BACKGROUND

Recent technological and scientific progresses have made it possible to develop Advanced Therapy Medicinal Products (ATMPs), including medicinal products that contain viable human cells that, by engrafting in patient's tissues, can restore specific physiological functions that are deficient in the patient. These innovative products, also called Cell Therapy Medicinal Products or Cell-Based Medicinal Products (CTMPs or CBMPs; Vamvakas S et al., 2011) may provide an effective alternative to the transplantation of organs, whose availability is severely limited and that present poor tolerance to cryopreservation. CBMPs may palliate these limitations as they rely on in-vitro cell expansion and on the cryopreservation of bulk cell products before being validated and used in appropriate clinical settings.

As of today, innovative medicinal products like CBMPs are unlikely to benefit from a vast manufacturing network since they contain living cells, are still at an early stage of development and are subject to highly demanding legal and regulatory frameworks.

Additionally, CBMPs are more difficult to distribute to patients than well-established small molecule-/protein-based medicinal product since they have short shelf lives of few hours, are highly sensitive to temperature, must be kept sterile at every stage of manufacturing since administrated parenterally (by injection or infusion), and requires high know-how and quality control standards (Ancans J, 2012; Belardelli F, et al., 2011; Salmikangas P and Celis E, 2011; Sekiya E, et al., 2012; Romagnoli L, et al., 2011; Sensebe L, et al., 2011; Gee A, 2009). Thus, it is important to formulate CBMPs as close as possible to the sites where patients are treated and deliver CBMPs to any site within a few hours in a predictable, safe and economically sustainable way.

These limitations should be taken into account when coordinating the activities and the overall organization at both bulk CBMP manufacturing facility and the medical facility for guaranteeing the sterility of CBMP formulation. In particular, cell processing systems have been recently designed and commercialized for separating, manufacturing, and/or formulating cell preparations and other biological products within closed systems that allow establishing sterile conditions in a more or less automated manner like Sepax (Biosafe), Elutra Cell Separation System (CardianBCT), or CliniMACS Prodigy (Miltenyi Biotec). This equipment may allow formulating CBMPs directly at the hospital. However, safety requirements for administering CBMPs to patients imply the availability of a cleanroom, or the use of an aseptic closed formulation system, in a temperature-controlled environment that also allow performing immediately the in-process quality control. Such facilities and equipment are not generally available at (or near) the medical site where the patient is treated. This problem consistently slows down CBMPs clinical development and wider adoption of CBMPs-based therapies, such as for establishing allogeneic unmatched cell therapies as much more pharmaceutical-like “off-the-shelf” products (Brandenberger R, et al., 2011; Foley L and Whitaker M, 2012).

Examples of modular laboratory or pilot production facilities for cell therapy and biological products have been described in several prior patent publications including French Patent Publication FR2819536; International Patent Publication WO93/25657; International Patent Publication WO03/0024312; International Patent Publication WO03/095765; International Patent Publication WO07/067656; International Patent Publication WO11/022325; US Patent Publication No. 2012/0077429; International Patent Publication WO07/105844; and International Patent Publication WO08/018671. Even if such structures can be established and adapted to a given use more quickly than traditional, bricks-and-mortar buildings, none of them have the required flexibility to transport bulk CBMP preparations, equipment and consumables (as those required by closed systems for processing biomaterials), and to perform all the procedures required to provide CBMPs to a medical site on short notice and according to regulatory requirements. Known facilities do not allow distributing CBMP formulations in a safe and effective manner not only at a specific medical site where one or more patients receive the CBMP-based treatment for a given time (e.g. up to a few days), but also at several, distinct medical sites within a given time frame (e.g. up to a few weeks) in a single distribution campaign, without returning to the bulk CBMP manufacturing facility.

### SUMMARY

The present disclosure provides a novel facility for formulating CBMPs in sites where said product is administered to a patient and that are distinct and away from the site where a bulk CBMP preparation is initially prepared and validated according to Good Manufacturing Practices. Such a facility, hereafter named as Mobile CBMP Formulation Facility (MCF Facility) allows an efficient access and distribution of CBMPs in medical sites in particular at those devoid of equipment, personnel, and/or areas dedicated to similar activities.

According to the teachings of the present disclosure, a facility for formulating a Cell-Based Medicinal Product, in particular at a site where said product is administered, is provided. An example facility includes

(a) A First Unit (Unit 1) that is a working space designed and equipped to allow operators to store, count, test, sample, formulate, and/or package cell preparations and produce Cell-Based Medicinal Product formulations according to Good Manufacturing Practices;
(b) A Second Unit (Unit 2) that is adjacent to and communicates with Unit 1 and allows operators to access Unit 1, to store the equipment and the materials, to prepare themselves before and after accessing Unit 1, and to bring Cell-Based Medicinal Product preparations, the equipment, and the materials in and out of Unit 1;
(c) A Third Unit (Unit 3) that is adjacent to Unit 1 and that houses the equipment for exchanging of electric power, air, and/or data with Unit 1, Unit 2, and/or Unit 4 by means of ports or any other connections; and
(d) A Fourth Unit (Unit 4) that is a vehicle allowing the transportation of a unitary structure comprising Unit 1, Unit 2, and Unit 3.

These four Units can be differently made accessible and assembled within MCF Facility (see FIGS. 1A-B and FIGS. 2A-D). In particular, each of Unit 2 and Unit 3 can be accessed by operators (by means of a door) from the exterior and/or from Unit 4. Moreover, an additional Unit 5 can be positioned within the unitary structure comprising Unit 1, Unit 2, and Unit 3, and in particular it can be positioned between, and communicate with (by means of doors), Unit 1 and Unit 2 only.

In a specific embodiment, Unit 1 comprises two or more Working Areas, each one dedicated to activities for formulating and validating CBMPs. In particular, Unit 1 comprises:

(i) A First Working Area that is equipped with a vertical laminar air flow connected to Unit 3 and with instruments for manipulating, sampling, formulating, and/or packaging of Cell-Based Medicinal Products;
(ii) A Second Working Area that is equipped with instruments for counting and/or testing cell preparations; and
(iii) A Third Working Area that is equipped for storing cell preparations and/or equipment to be used within Unit 1.

Within this embodiment, Unit 1 is specifically designed and operated according to Good Manufacturing Practices. At this scope, Unit 1 comprises a First Working Area can be equipped with closed systems that allow establishing sterile conditions for formulating CBMPs, and/or with a glove-box. A Second Working Area can be also equipped with a computer for monitoring, storing, elaborating, and transmitting data that are generated by operators and equipment within Unit 1. A Third Working Area can be also equipped with furniture for storing consumables, plasticware, waste, water, and formulation media at appropriate temperature (see FIGS. 3A-E).

In some examples, the walls of at least Unit 1, and also of the Units that allow accessing operators to Unit 1 (i.e. Unit 2 and, when present Unit 5) are made of composite panels comprising:

(a) A water- and air-tight inner surface;
(b) An intermediate thermally insulating material; and
(c) A shock-resistant, water- and air-tight external surface.

The assembly of these materials and elements should allow the isolation of equipment, materials, reagents, and operators within such Units from the external environment while the Facility is moving or when at a given location, so that the resulting internal environment make possible to store, validate and formulate CBMPs, as well as to retrieve and store any relevant data on the operations within the MCF Facility, in the most appropriate conditions.

In particular, the water- and air-tight inner surface should be sleek and smooth so that equipment, furniture (e.g. non-/foldable tables, laboratory benches cabinets or multipurpose drawers), containers, and elements within the Units can be easily moved and the surface can be washed in accordance with the Good Manufacturing Practices. The intermediate thermally insulating material can be rigid or flexible, in various formats (slabs, sheets, blocks, tiles), and comprising high density rock or glass wool or other foam or sponge material (e.g. polyurethane) that is capable to limit the passive exchange of thermal energy with the external environment.

In some examples, MCF Facility can be also equipped with an HVAC system, which in some examples is located within Unit 3, that exchanges air and regulates temperature in Unit 1 and, optionally, Unit 2 and/or, when present, Unit 5 (e.g. through grids). The HVAC system allows exchanging air and heat in an highly controlled manner to maintain temperature, humidity, pressure and cleanliness of the air at required level, in particular it can be equipped with filters that can make Unit 1 (or at least the First Working Area) compliant with at least ISO 14644-1 Class 7 standard.

In some examples, MCF Facility can be also equipped with instruments (such as sensors, computers, touch screens, and other devices) for acquiring, monitoring, storing, elaborating, and transmitting data related to the preparation of CBMPs formulations, the manipulations made in MCF Facility, and the status of each Unit, Working Area, and/or equipment in MCF Facility. In particular, Unit 1, Unit 3 and/or Unit 4 can contain equipment for acquiring, storing, elaborating, and/or transmitting said data, for example to other facilities outside MCF Facility.

In some examples, MCF Facility, and Unit 1 in particular, allow operating according to ISO standards that are applicable to production, formulation, administration, or medical use of Cell-Based Medicinal Product, formulations, In particular, MCF Facility should be equipped to allow operators formulating and validating a Cell-Based Medicinal Product comprising cells, such as for example cells of human origin, such as stem or progenitor cells, primary cells, or cells that have been genetically modified.

In some examples, MCF Facility can be moved in different locations by means of a Unit 4, being a vehicle (e.g. a truck or any other type of self-propelled vehicle) for transporting the unitary structure comprising Unit 1, Unit 2, and Unit 3 (and Unit 5, when present), that can be designed, equipped, assembled, and operated to satisfy all technical, legal, and regulatory requirements (in particular Good Manufacturing Practices) of the site in which MCF Facility is moved and of the public transportation infrastructures.

The present disclosure also provides a method of providing formulations of a Cell-Based Medicinal Product in a site where said formulations are administered to a patient. An example method includes:

(a) Building a unitary structure comprising equipment and working spaces that allow operators to store, formulate, and validate Cell-Based Medicinal Products according to Good Manufacturing Practices, and assembling said structure with a vehicle for establishing a facility;
(b) Providing bulk Cell-Based Medicinal Products that are prepared according to Good Manufacturing Practices;
(c) Storing said bulk Cell-Based Medicinal Product preparations within the facility; and
(d) Moving the facility to a site where said bulk Cell-Based Medicinal Product preparations are used for producing and validating formulations of Cell-Based Medicinal Product within said facility, and where said formulations are administered to a patient.

CBMP formulations that are provided according to this method comprises cells, such as, for example, of human origin, such as stem or progenitor cells, primary cells, or cells that have been genetically modified for cell therapy in general, and in particular allogeneic, somatic cell-based therapy. Moreover, these CBMP formulations satisfy all technical, legal, and regulatory requirements (in particular Good Manufacturing Practices) of the site in which the facility is moved, as well as specific requirement that is associated to the status of the patient in connection to the administration of cell products for allogeneic, somatic cell-based therapy.

The Detailed Description and the Figures provide additional details on the teachings of this disclosure, and in particular on further examples that are associated to alternative MCF Facilities that are specifically assembled, connected, operated, and/or equipped.

### DESCRIPTION OF FIGURES

FIG. 1A shows a top view of an example physical arrangement of example Units within an exemplary MCF Facility. In the top view (FIG. 1A), doors for the access of operators for performing CBMP Formulation and for MCF Facility maintenance, control and, and transport, as well as exchanges of materials, air, electric power or data within MCF Facility or with exterior (Ext.), before, while, and/or after it is moved to a site by means of Unit 4 (the position of its front end is indicated), are also shown (see description of symbols below the drawing). Temperature and air control within Unit 1 (directly) and Unit 2 (indirectly) is made possible by means of HVAC equipment located in Unit 3 and grids for air flow. Connections for data exchange and electric power supply across the Units are indicated externally but are actually made possible by circuits that are assembled within MCF Facility.

FIG. 1B shows a side view of the example of FIG. 1A. In the side view (FIG. 1B), physical arrangement of a Unit 4 (a truck, for example) that is assembled with a unitary structure comprising Unit 1, Unit 2, and Unit 3 is shown.

FIG. 2A shows an example alternative arrangement of example Units within an example MCF Facility. In a first top view (FIG. 2A), Unit 3 (instead of Unit 2, as in FIG. 1A) is positioned between Unit 1 and front end of Unit 4 with which it can be communicating with.

FIG. 2B shows another example alternative arrangement. In a second top view (FIG. 2B), Unit 3 and Unit 1 are positioned so that are both adjacent to Unit 2 that can be also adjacent, and (if required, communicating with) the front end (and/or, if appropriate, the rear end) of Unit 4.

FIG. 2C shows yet another example alternative arrangement. The arrangement of Units can be modified to include Unit 5 between Unit 1 and Unit 2 (top view in FIG. 2C), adapting accesses and circuits for exchanges of materials, air, electric power or data within MCF Facility or with exterior consequently. Symbols and definitions are the same of FIG. 1A.

FIG. 2D shows still another example alternative arrangement. The example arrangement of FIG. 2D also includes Unit 5 between Unit 1 and Unit 2 (top view in FIG. 2D).

FIGS. 3A-E show exemplary Unit 1 and related Working Areas. The top view (FIG. 3A) shows respective positions of First (1.1), Second (1.2), and Third (1.3) Working Areas of Unit 1 and of two operators, one producing final CBMP formulation (Op. 1) and one performing the Quality Control of final CBMP formulation (Op. 2). Side views of these working areas that are available to the operator when performing Quality Control (FIG. 3B) or CBMP formulation (FIG. 3C) are shown. Specific areas can be dedicated to the storage of bags containing bulk CBMP preparations in liquid nitrogen (1.3.A), the storage of temperature-sensitive media and consumables at 4° C. (1.3.B), the storage of temperature-insensitive consumables and equipment (1.3.C), the storage of waste that is generated when producing CBMP formulation or performing Quality Control (1.3.D), the technical space for providing the laminar air flow and light to the operator when producing CBMP formulations (1.1. A), and the working space where operators either produce CBMP formulations (1.1.B) or perform Quality Control (1.2). Top views of exemplary equipment (white boxes) and their arrangement with ports (black boxes) into 1.1.A of First (FIG. 3D) and Second Working Area (FIG. 3E) are shown. Control panels and touch screens (e.g. for temperature, air flow or illumination) can be located in any Working area and/or on the walls of Unit 1.

FIG. 4 is a flowchart summarizing example elements and example steps for providing formulations of Cell-Based Medicinal Products by means of an MCF Facility at a given site that is equipped and operated for treating patient with CBMP (Medical Facility). Cryogenized bulk CBMP preparations are prepared according to Good Manufacturing Practices in a bulk CBMP Manufacturing Facility and then transferred within bags to a specific area of MCF Facility (1.3.A; see FIG. 3A) where they are stored during transit to the Medical Facility and until use. Distinct spaces within MCF Facility are also dedicated to the storage of Formulation Media (1.3.B; see FIG. 3B) and other consumables (1.3.C; see FIG. 3B) during the transit of MCF Facility to the Medical Facility and until use. When on site, a bag of cryogenized bulk CBMP preparation that is stored in a freezer or a liquid nitrogen tank, is used to produce a CBMP formulation in a dedicated space of MCF Facility (1.1.B; see FIG. 3C and FIG. 3D). This formulation is sampled and then tested on site for in-process Quality Control within another space of MCF Facility (1.2; see FIG. 3B and FIG. 3E). Some of these samples may be stored as retention samples in MCF Facility (1.3.B; see FIG. 3B) and, if needed, tested elsewhere later on (e.g. at original bulk CBMP Manufacturing Facility or at the Medical Facility). CBMP formulations are then transferred at the Medical Facility where it is used for treating patients. Data that are acquired, elaborated, and stored in MCF Facility can be exchanged also with other, remote facilities.

### DETAILED DESCRIPTION

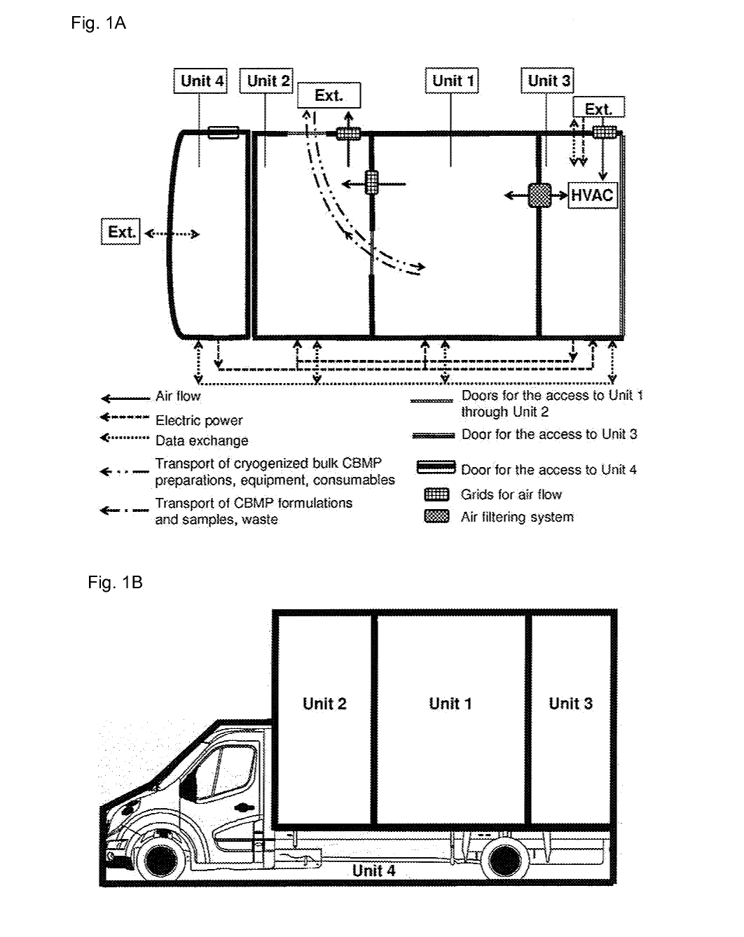

The present disclosure provides an example facility (MCF Facility) that combines the choice and the design of building elements, the means for storing and/or distributing materials and utilities, and commercially available equipment for producing CBMP formulations in an innovative and flexible manner. MCF Facility is assembled in Units that can be differently equipped and arranged (as exemplified in FIGS. 1A-3E) for providing timely and efficiently CBMP formulations to one or more medical sites that are equipped and knowledgeable for administering CBMP formulations but not for producing such CBMP formulations in GMP conditions.

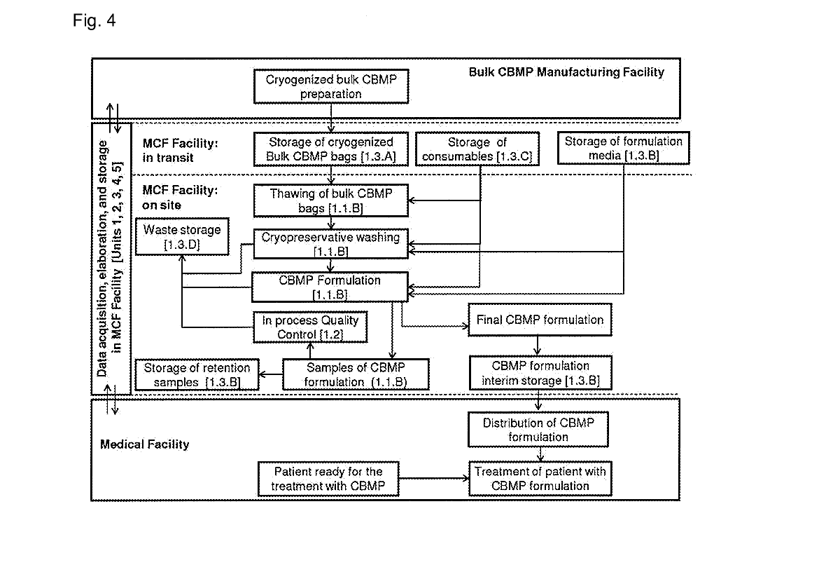

The combination of structural features and equipment within MCF Facility makes it possible to provide CBMP formulations “on demand” by using CBMP preparations that were produced and pre-validated according to the Good Manufacturing Practices (especially in relation to dosage, reproducibility, temperature, and sterility). This CBMP formulation can be finally administered in coordination with a local medical team in charge of patient's treatment (FIG. 4) which, in absence an appropriate facility that can be quickly relocated and made fully operational on short notice, would be hardly capable to offer such a treatment on their own.

The expression “Good Manufacturing Practices” (GMP, also referred to a cGMP or “current GMP”) refers to recommendations and guidelines with respect to manufacturing and formulation of drugs and biologies that are established and implemented by national or international competent agencies worldwide, such as WHO (World Health Organization), FDA (Food and Drug Administration) in USA, Indian Council of Medical Research, or EMA (European Medicines Agency) in Europe.

Specific GMP regulations are issued for specific products such as CBMPs, regularly reevaluated and updated by specific committees, and published on the websites of such agencies and reviewed in the literature (Belardelli F, et al., 2011; Romagnoli L, et al., 2011; Salmikangas P and Celis E, 2011; Sekiya E, et al., 2012; Sensebe L, et al., 2011). Since GMP-compliant procedures may change, the structural features and the equipment of MCF Facility can be adapted in accordance with GMP requirements of the relevant regulatory agency.

The GMP specifications for Advanced Therapy Medicinal Products (ATMPs), and for CBMPs in particular cover a large of topics and parameters that need to be evaluated and established for designing, assembling and operating MCF Facility correctly. The major topics covered in GMP specifications (EBE Concept Paper, 2010; European Commission, 2009) include Quality Management (e.g. responsibilities, external/internal audits, documentation, product quality review before and after official release), procedures applied by operators (e.g. safety, hygiene, and related training), design and construction of facilities (e.g. utilities, storage area, containment, flow of material, personnel, and waste), equipment (e.g. type, maintenance, calibration, instructions), materials (e.g. management, general controls and requirements, traceability), production activities (e.g. cell bank and maintenance, protocols for cell culture, harvesting/isolation/purification of cells, procedures for viral removal/inactivation), formulation/packaging activities (e.g. buffer and media preparation, in-process quality controls, containers, labelling/packaging materials), storage and distribution (e.g. logistics, security, import/export procedures), and laboratory controls for validation (e.g. testing, analytical procedures, decontamination/cleaning procedures, equipment, asepsis/sterility, certificates of analysis, stability/shelf-life monitoring, retention samples, manufacturing/preservation policies).

MCF Facility allows operating according to ISO standards that are applicable to production, formulation, administration, or medical use of cell-based products. Specific ISO standards that can be applicable to equipment present in MCF Facility and related procedures include ISO 13022 (Medical products containing viable human cells—Application of risk management and requirements for processing practices), ISO 14644 (Cleanrooms and associated controlled environments), ISO 13408 (Aseptic processing of health care products), ISO 10993 (Biological Evaluation of Medical Devices), ISO 9001 (Quality Management Systems), ISO 151 89 (Medical laboratories—Requirements for quality and competence), ISO 13485 (Medical devices—Quality management systems—Requirements for regulatory purposes), ISO 14971 (medical devices—Application of risk management to medical devices), ISO 14001 (Environmental management systems), ISO 11737 (Sterilization of medical devices), and/or ISO 14160 (Sterilization of health care products). In particular, Unit 1, and more particularly First Working Area, should be a working space that has a low level of environmental pollutants such as dust, virus, bacteria, pollen, or aerosol particles. Different official standards indicate reference values for a controlled level of contamination that is specified by the number of particles per cubic meter at a specified particle size.

Table I provides ISO 14644-1 (2010) standards and corresponding classes

| Maximum | Class | | | | | | | | |
|---|---|---|---|---|---|---|---|---|---|
| Particles/m³ | ISO 1 | ISO 2 | ISO 3 | ISO 4 | ISO 5 | ISO 6 | ISO 7 | ISO 8 | ISO 9 |
| ≧0.1 μm | 10 | 100 | 1,000 | 10,000 | 100,000 | 1.0 × 10⁶ | 1.0 × 10⁷ | 1.0 × 10⁸ | 1.0 × 10⁹ |
| ≧0.2 μm | 2.37 | 23.7 | 237 | 2,370 | 23,700 | 2.37 × 10⁵ | 2.37 × 10⁶ | 2.37 × 10⁷ | 2.37 × 10⁸ |
| ≧0.3 μm | 1.02 | 10.2 | 102 | 1,020 | 10,200 | 1.02 × 10⁵ | 1.02 × 10⁶ | 1.02 × 10⁷ | 1.02 × 10⁸ |
| ≧0.5 μm | 0.352 | 3.52 | 35.2 | 352 | 3,520 | 35,200 | 3.52 × 10⁵ | 3.52 × 10⁶ | 35.2 × 10⁶ |
| ≧1 μm | 0.0832 | 0.832 | 8.32 | 83.2 | 832 | 8,320 | 83,200 | 0.8 × 10⁶ | 8.32 × 10⁶ |
| ≧5 μm | 0.00293 | 0.0293 | 0.293 | 2.93 | 29.3 | 293 | 2,930 | 29,300 | 0.2 × 10⁶ |

Alternatively, Table II provides GMP EU standards and corresponding classes

| Maximum Particles/m³ | | Class | | | |
|---|---|---|---|---|---|
| (by size) | | A | B | C | D |
| ≧0.5 μm | At rest | 3,520 | 3,520 | 352,000 | 3,520,000 |
| ≧5 μm | | 20 | 29 | 2,900 | 29,000 |
| ≧0.5 μm | In operation | 3,520 | 352,000 | 3,520,000 | n/a |
| ≧5 μm | | 20 | 2,900 | 29,000 | n/a |

Thus, MCF Facility should be equipped and operated to establish appropriate ISO and non-ISO GMP standards in specific areas, and in particular in areas where bulk CBMPs preparations in order to obtain CBMP formulations that can be transferred from MCF Facility to the medical team in charge of treating the patient accordingly.

In the some examples, a facility (the MCF Facility) is provided that comprises at least four Units (Unit 1, Unit 2, Unit 3, Unit 4) that can be alternatively designed, assembled, arranged, equipped, connected, and/or operated as exemplified in FIGS. 1A-4 and in the following paragraphs that, without limitation, describe further examples of the teachings of the present disclosure.

Unit 1 is the core structure where CBMP formulations are actually produced, all equipment needed for such operations is stored and used, and GMP specifications should be more strictly applied according to the requirement of the competent regulatory agency. Unit 1 includes specific physical and functional association with Unit 2 and Unit 3 that are adjacent and form with Unit 1 (with or without a further Unit 5) a unitary structure.

Unit 2 represents an example passage to Unit 1 from the external environment and allows operators to prepare themselves before and after accessing Unit 1 from exterior and to bring materials in and out of Unit 1 (with or without passing through an intermediate Unit 5), as well as to store equipment and materials that are not immediately needed in Unit 1 and that can be stored outside Unit 1 (e.g. documentation, cleaning/decontaminating materials, water or liquid nitrogen reservoirs, clean room garments, or other laboratory clothing). Thus, Unit 2 is also designed and operated according to GMP and further separated into two distinct areas, one required for the passage and activities of operators, and another for storing which can be physically separated by a door or formed by cabinets or multipurpose drawers.

Unit 3 houses the equipment that is connected to Unit 1 (and, optionally Unit 2, Unit 4, and/or Unit 5), by means of ports or any other connecting means for exchanging electric power, air, data, and/or other utilities. Unit 3 can be accessed by operators occasionally for controlling or installing HVAC, batteries, connections, circuits, plugs, reservoirs, control panels, and any other equipment for operating correctly MCF Facility on transit or on site.

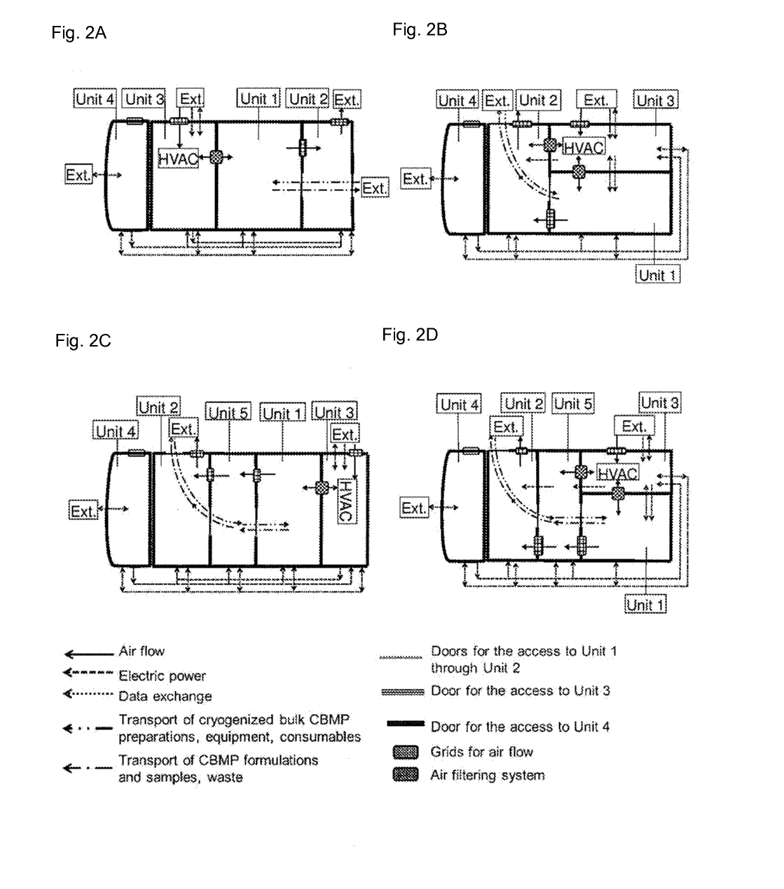

The unitary structure comprising Unit 1, Unit 2, and Unit 3 (with or without a further Unit 5) is then built in and associated to a Unit 4, that is a vehicle (such as, for example, a truck), allowing the transportation of the unitary structure, as well as of operators, consumables, equipment and other materials. In some examples, Units can be assembled to have Unit 2 positioned between the front end of Unit 4 and Unit 1, with Unit 3 between Unit 1 and the rear end of Unit 4 (FIG. 1B) but alternative arrangements can be appropriately established, combined, and adapted (e.g., FIGS. 2A-D). Even though FIGS. 1A-B and FIGS. 2A-D show that the unitary structure comprising Unit 1, Unit 2, and Unit 3 (with or without a further Unit 5) can be accessed directly from the exterior from Unit 2 and/or Unit 3, these two Units can be also communicating with the adjacent front or the rear end of Unit 4 by means of a door or other passage that can be used as additional (or alternative) access of operators within MCF Facility when on transit or on site.

When Unit 5 is present, the arrangement of Units within the structures as described above can be modified and adapted consequently (FIG. 2C and FIG. 2D). The optional Unit 5 is intended to provide a space intermediate between GMP-validated Unit 1 and Unit 2 that communicating by means of a door with exterior directly (or, indirectly, through Unit 4), where specific conditions can be established, in particular for establishing specific ISO compliancy distinct from those of Unit 1 and Unit 2.

For instance, when a specific Unit 3 (comprising an air filtering system with related grids and connections to other Units) is combined with appropriate hermetic, airtight sealing (and, possibly, sensors) for keeping temperature, air purity or pressure under control in Unit 1 and Unit 5, it can be possible to establish an MCF Facility (in particular in the arrangement shown in FIG. 2D) with Unit 1 being compliant with at least ISO 14644-1 Class 5 (or GMP EU class B) and Unit 5 being compliant with at least ISO 14644-1 Class 7 (or GMP EU class C).

These values can be achieved by making use of air filtering system that is coupled to systems for controlling air temperature and air recycling, usually defined as HVAC (heating, ventilation, and air conditioning) that include High-efficiency particulate air (HEPA) filters. In some examples, this equipment is in Unit 3 and receives air from exterior (e.g. through a grid), adapting air temperature and/or humidity according to requirements (e.g. at 20° C.), pushing air through appropriate filters such as HEPA (High-Efficiency Particulate Air) or ULPA (ultra-low penetration air) filters, and employing laminar or turbulent air flow principles for finally directing air to Unit 1, and, depending from the arrangement of Units, directly or indirectly to Unit 2 and/or Unit 5 (and possibly recycling air within said Units).

Regarding the dimension of MCF Facility, Units can be constructed in different dimensions but the specific limitations may be associated to both the choice of Unit 4 and the actual permanent presence of one or more operators within a given Unit and the size of equipment they need for their activities herein. For instance, Unit 1 can be the larger one (e.g. with a floor surface of at least 4 m² and high at least 2 m) since it should allow housing one or more operators for certain number of hours and all equipment that is required for formulating and validating CBMPs in GMP conditions. Unit 2 and, if present, Unit 5 can be smaller (e.g. with a floor surface of at least 2 m² and high at least 2 m) since both Units allow one or more operators preparing themselves before accessing Unit 1 and leaving MCF Facility, as well as bring in and out materials and possibly store some materials during the transit of MCF Facility or on site. Unit 3 can be the smallest one (e.g. with a floor surface of at least 1 m² and high at least 1 m) since it is dedicated only for housing equipment, without requiring (in general) the permanent presence of at least one operator within Unit 4 while MCF Facility is in transit or on site.

Consequently, the unitary structure comprising Unit 1, Unit 2, Unit 3, and, if present, Unit 5, may have a floor surface at least 7 m², 9 m², 11 m² or more and a height of at least 2 m, 2.5 m, 3 m or more. However, depending on the actual construction blueprint, the presence or not of Unit 5, and the required flexibility in both use and transport of MCF Facility, other larger dimensions can be chosen and MCF Facility may be built accordingly. In some examples, such as when Unit 4 is a truck, Unit 1 may have a floor surface of at least 6 m² and be high at least 2.5 m, Unit 2 (and, if present, Unit 5) may have a floor surface of at least 4 m² and an height of at least 2.5 m, and Unit 3 may have a floor surface of at least 2 m² and an height of at least 2.5 m. Once assembled on a truck as Unit 4, the MCF Facility may not exceed, in some examples, a width of 3 meters, a height of 3 meters, a length of 7 meters, and a weight of 3.5 tons.

Regarding the choice of materials for constructing the framework and the external parts of MCF Facility, the unitary structure comprising Unit 1, Unit 2, Unit 3, and, if present, Unit 5 should be adapted for resisting to physical stress (due to combined presence of one or more operators, and possibly heavy equipment, with transportation in more or less standard locations). Moreover, this structure should be water-air-tight and with appropriate sensors and devices for establishing temperature control and sterility (i.e. complete absence of viable microorganisms or virus), such requirements applying in particular to Unit 1 where most GMP-associated activities are performed. This structure can be built in metal, e.g. a steel or aluminum frame, with an all steal or aluminum walls and doors, with composite panels separating the Units as described above, with glass or transparent plastic elements (when needed) to inspect activities, and is stably associated with Unit 4.

Regarding the choice of materials for constructing the internal parts of MCF Facility, all the floors, windows, walls, ceiling, and any additional surface (in particular within Unit 1) where equipment or operators are permanently or occasionally positioned (e.g. laboratory benches, furniture, or chairs) should be selected and designed for maximal cleanability using products that are adapted for GMP facilities, with minimal or no cracks, holes, and other spaces in which dust or bacteria may accumulate. Any window or door is flush-mounted to the interior to allow effective cleaning. Floors, ceiling and walls are covered with an appropriate synthetic material (e.g. an epoxy resin). Such materials, as well as the cleaning/decontamination products and procedures to be used in an environment for CBMP production or formulation, should be compliant with GMP specifications as described by FDA, EMA or equivalent national or international regulatory agency.

Regarding the access to each Unit, it is made possible by doors whose number, position, and other features depending on the arrangements of the Units for operating correctly MCF Facility. In particular, the operators can bring all relevant materials in and out of Unit 1 and Unit 3 from exterior (and/or, optionally, from Unit 4) or after passing through Unit 2 (and, optionally, Unit 5). Moreover, depending on the actual vehicle that is selected, the access to Unit 2 and/or Unit 3 can be possible only from exterior and/or specific areas within Unit 4 (e.g. the front or the rear end).

In some examples, Unit 1 has a single door to permit the passage of operators and materials from Unit 2. Unit 4 may have, in addition to the door for the access from the exterior, one or two further doors for accessing (even when MCF Facility is in transit) Unit 2 and/or Unit 3, at the front or at the rear end. Unit 2, in some examples, has two doors, one for communicating with Unit 1 and the other one for communicating with the exterior or with Unit 4, but an additional door may allow communicating with Unit 3, if adjacent to Unit 2. Unit 3 has to accessible by at least one door, communicating with exterior, Unit 2, or Unit 4. When present, Unit 5 has two doors, one communicating with Unit 1 and one with Unit 2. In general, the doors giving the access to Units, and in particular giving the access to Unit 1 (and, when present to Unit 5) are, in some examples, built and assembled to be airtight and watertight for transporting and formulating appropriately CBMPs (e.g., in connection to air quality, maintenance of equipment, or storage of materials).

The structure that is formed by Unit 1, Unit 2, Unit 3, and, optionally, Unit 5 has different supplies in order to be operational when in transit or on site, as summarized in FIGS. 1A-3E. In particular, Unit 3 provides a series of services, e.g. by housing the equipment for supplying air (i.e. HVAC system) and electric power and, if needed, any other utility that should be provided by ports and other connection means inside Unit 1 and, optionally, Unit 2 and/or Unit 5 such as gases (oxygen), liquid nitrogen, or water. These utilities are obtained from the exterior (or from Unit 4, if adequately equipped and in particular when in transit) and, if possible, stocked in Unit 3 and/or Unit 4 (e.g. by means of batteries, tanks, or appropriate reservoirs).

Thus, when MCF Facility is on site, Unit 3 and/or Unit 4 can be plugged to public or private electricity, water, and or gas networks, in addition to one or more grids for the air input (and optionally output) for HVAC system. Alternatively, the MCF facility can be adapted to be entirely autonomous and thus unaffected by specific conditions (e.g. power voltage and gases composition), which contribute to the process consistency in different medical sites (according to local or national standards). At this scope, electric power generator, water containers, and reservoirs for gases and other utilities can be assembled within Unit 3 and/or Unit 4.

Unit 3 can also house computers, touch screens, control panels, and other electronic instruments that are connected to instruments, sensors, and other devices into any of Units and that can exchange data with them, as well with distant sites using wired or wireless technologies such as mobile phones, Internet protocols, Bluetooth, GPS, radio waves, or Wi-Fi. When moving or operating MCF Facility, these data exchanges can be performed directly and autonomously between such distant sites and Unit 3 and/or controlled by operators and/or by other electronic devices that are located in any other Unit, in particular in Unit 4 or in Unit 1.

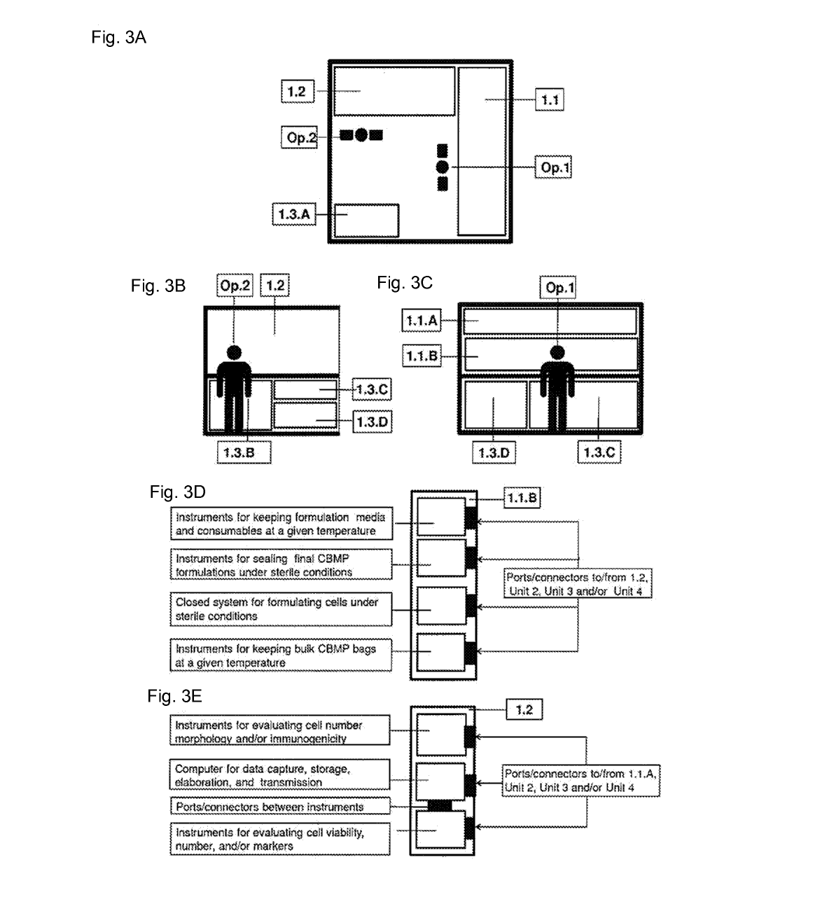

Regarding ports and connectors, they can be of different types (for electric power, data transmission, water, waste (e.g. cell culture medium, water, or biological materials), gases, or air filtration inputs/outputs), and in appropriate positions and number for establishing efficient standard procedures for formulating and validating CBMPs in MCF Facility (FIGS. 1A-B and FIGS. 2A-D). At least one Working Areas within Unit 1 may comprise ports and/or connectors that allow supplying electric power and providing input/output of data, air, or water towards devices in the same Unit and/or in Unit 3 (FIGS. 3A-E). Both the First Working Area (1.1.B) and the Second Working Area (1.2) may comprise a space where the devices can be connected to the ports and connectors (and, when needed, directly to each other). Ports for electric power supply should be connected to a circuit and equipment that allow illuminating Unit 1 and running equipment such freezers, refrigerators, temperature-controlled water baths vacuum/water pumps, and electronic devices alternatively with MCF Facility autonomous electric power source (e.g. in Unit 4 or Unit 3) while it is on transit and with the local electric power grid (if available or compatible) while MCF Facility is on site. Moreover, since CBMPs are actually formulated in GMP conditions under a laminar air flow in Working Area 1.1.B (FIGS. 3C and 3D), a dedicated technical area can be present on the top of Working Area 1.1.B (see 1.1.A in FIG. 3C) for providing the desired air flow there and in the rest of Unit 1, as well other functions that may be required (e.g. light). Working Area 1.1.B can be further isolated from the rest of Unit 1 by a glove-box that is operated from outside.

Regarding the type and arrangement of devices for monitoring the status of MCF Facility and of operations that are performed within (on site or remotely), sensors, video or photo cameras, and any other appropriate instrument can be used for monitoring and possibly transferring the data and/or images that have been acquired in specific locations to instruments in Unit 3, Unit 4, and/or any other different location that may trigger further actions automatically (e.g. adapting temperature or alerting operators that an equipment is not working or located properly). In particular, appropriate sensors may register air quality (by indicating concentration of specific gases or the concentration of particles below or above a given size), air pressure, security problems, chemical or biological contamination, temperature, or humidity within any of Unit 1, Unit 2, Unit 5, and/or Unit 3. If monitored values are not those pre-established, sensors may alert operators by triggering the automatic call of pre-programmed emergency telephone numbers and/or by sending communication via text or e-mail.

Regarding the type of equipment in Working Areas of Unit 1, they should be appropriately distributed in the different Working Areas for completing all basic operations for storing, counting, testing (e.g. cell viability), sampling, formulating, and/or packing a cell preparation to produce Cell-Based Medicinal Products according to Good Manufacturing Practices.

The First Working Area (FIG. 3C and FIG. 3D) is equipped with a vertical laminar flow and a laboratory bench with sufficient surface to operate (e.g. a cleanable, metal surface with or without small holes allowing air flow from the top and air recycling) onto which instruments for manipulating, sampling, formulating, and/or packaging cell preparations under sterile conditions (i.e. using aseptic techniques for handling or processing cell preparations where the risk of contamination with living or dead bacteria, fungi or viruses and other biological agents is minimized or prevented) are positioned appropriately. Such equipment may comprise commercial closed systems for separating, manufacturing, and/or formulating cell preparations and other biological products under sterile conditions in a more or less automated manner like Sepax, Elutra Cell Separation System, or CliniMACS Prodigy, pumps, incubators for cooling/heating materials at a given temperature, a sealing unit (e.g. Hematron, Fenwal Inc.), sensors, and other auxiliary equipment that can be validated for use in GMP conditions.

The Second Working Area (FIG. 3B and FIG. 3E) is dedicated to the validation of CBMP formulations following their preparation and sampling in the First Working Area, in particular with the scope of identifying any contaminant (i.e. impurity of a chemical or microbiological nature, or foreign matter that have been unintentionally introduced into CBMP formulation during production, sampling, packaging, storage, or transportation). It is equipped with a laboratory bench with sufficient surface to operate (e.g. a cleanable, metal or plastic surface) onto which instruments for counting or otherwise measuring and/or testing, cell preparations are positioned appropriately, as well as a computer for acquiring, elaborating, storing, and transmitting data to enable a satisfactory control of GMP operations. Such equipment may comprise commercial instruments for concentrating/washing CBMP samples (e.g. centrifuges or waste liquid pump associated to an appropriate pipe and vessel), for evaluating cell morphology and/or number (e.g. microscopy for optical and/or fluorescence-based observations, with or without a camera) or biological and functional features, including the direct or indirect quantification of relevant biological markers/contaminants (at the protein and/or DNA level) and/or chemical contaminants, enzymatic activities, immunogenicity, potency (i.e. quantitative measure of a biological activity based on those attributes of a CBMP that are linked to required therapeutic properties). Other auxiliary equipment used for quality control (e.g. for flow cytometry, microbial/viral sterility tests, gel electrophoresis, binding assays, immunoassays, karyotyping, cell viability, or nucleic acid amplification) may be also present.

A Third Working Area may be present and specifically dedicated to the storage of cell preparations (bulk CMBP preparations or final CBMP formulations), equipment, consumables (to be kept either at room temperature or at low temperature), and waste disposal containers (for separating liquid, solid, and/or biological materials). Distinct spaces can be defined within Third Working Area, and are, in some examples, positioned on or near the floor, for example below the laboratory bench of the First and/or Second Working Area or on the floor aside them (FIGS. 3B and 3C). Optionally, some of these spaces and equipment can be also positioned on the top of the laboratory bench of the First and/or Second Working Area. The respective volumes and overall arrangement of these spaces can be adapted according to the GMP specifications and the scheduled activities by equipping with cabinet, multipurpose drawers, and/or refrigerated/insulated devices (e.g. freezers, liquid nitrogen tanks).

The cell preparations to be used for producing CBMPs can be initially isolated from any tissue or organ (including liver, bone marrow, kidney, muscle, placenta, skin, pancreas, central/peripheral nervous system, lung, retina, or reproductive organs) or within cell preparations that are obtained from biological fluids (such as blood). Bulk CBMP preparations can be produced at an industrial scale in a GMP-validated facility (e.g. using bioreactors, membranes, microspheres, or any other technical solution for improving bioprocessing and cell expansion while maintaining desired cell properties). These CBMP preparations then can be transferred in MCF Facility for transporting them to a medical site as bags of cryopreserved CBMP preparations (each containing at least 10³, 10⁶, 10⁹, 10¹² cells or more) that are appropriately labelled. The cryopreservation of CBMP (i.e. its protection from damage that can occur during cooling and storing at very low temperatures, for example at −50° C., −70° C., or at even lower temperatures) can be performed using any appropriate a serum-containing or serum-free preservation medium (e.g. commercially available cryopreservation formulations) and/or in the presence of a cryoprotecting agent (e.g. dimethyl sulfoxide at an appropriate concentration).

Before being formulated into bulk CBMP preparations, the cells characterizing the CBMP (being adult or embryonic stem cells, progenitor cells, primary cells, or otherwise therapeutically useful cells, such as for example, of human origin, that have been expanded and/or modified using cell culture techniques) can be transiently or stably modified by exposing said cells to biological or chemical agents, or by introducing said agents within the cells (e.g. by treating cells with growth factors in cell culture and/or introducing nucleic acids that affect overall expression profile of the cells such as microRNAs or with retrovirus-based vectors). In this latter case, these cells can be genetically modified using conventional gene transfer methods. Following their transformation with appropriate vectors, the resulting cells can be provided as CBMP preparations expressing recombinant proteins or containing nucleic acids that allow said genetically modified cells to perform improved and/or additional therapeutic activities.

Regarding the transportation of MCF Facility, the structure comprising Unit 1, Unit 2, Unit 3, and, if present, Unit 5 may, in some examples, be built as a single, unitary, pre-fabricated module that is assembled with Unit 4 in a way that allows moving MCF Facility on standard roads and infrastructures from the bulk CBMP manufacturing facility to the medical site. Such structure can be adapted to be built in and transported with a Unit 4 being an automotive vehicle including a wheeled vehicle (e.g. a truck or a van), a helicopter, a cargo airplane, a train, or a ship. In particular, the MCF Facility, in some examples, is assembled in a dedicated truck, a small airplane, or a small boat that can make use of public infrastructures to allow rapidly distributing CBMPs across sites.

In some examples, the MCF Facility is equipped to provide means to immobilize and stabilize the vehicle when the CBMP is manufactured. For example, retractable external jacks may be added to wheeled vehicles to stabilize shock absorbers when the vehicle is parked. The MCF Facility also provides, in some examples, a means to secure the equipment, the materials, and the reagents during transportation. While small equipment, consumables, and other material can be safely stored in appropriate protective boxes secured with retention nets, larger equipment can be semipermanently fixed on the bench by means of metallic cradles, straps and hooks. In this way, the equipment is secured, can be rapidly set up and easily removed if needed (e.g. for maintenance or cleaning).

When on site, the bags of cryopreserved CBMP preparations, after being appropriately thawed, are used to produce the final CBMP formulation in the dedicated area of Unit 1 (Working Space 1.1. B; see FIGS. 3C and 3D). Depending on the desired treatment and labelling, the operator in Unit 1 can manipulate bulk CBMP preparations with the appropriate equipment for GMP standards for obtaining CBMP formulation that may differ in terms of cell concentration (e.g. at least 10³, 10⁶, 10⁹ cells/ml or more), volume (at least 0.1, 1, 10, 50 ml or more), type of container (e.g. tube or bag made of appropriate plastics or other polymers), form (e.g. suspension of isolated or aggregated cells, encapsulated cells, cells embedded within a matrix, a synthetic or natural scaffold), method of administration (e.g. intravenous or intraportal injection, implantation), formulation media, and addition of non-cellular compounds providing supplementary effects (e.g. drugs with immunosuppressive, anti-coagulant, or anti-inflammatory properties), labelling, or other additives, excipients, vehicles, and/or carrier that are compatible with cell-based pharmaceutical compositions that may include further specific buffers, growth factors, or adjuvants, wherein the amount of each component of the final CBMP formulation is defined (in terms of micrograms/milligrams, volume, or percentage).

The literature provides different protocols for generating CBMP formulations that can be adapted according to the approved clinical protocol, the officially authorized protocol for a clinical study, or other requirements established together with the team at the medical facility that may define additional criteria (e.g. related to the patient(s) to be treated on site and/or their equipment). In particular CBMP formulations can be provided in an appropriately labelled dosage for single administration, or for multiple administrations within a short period of time (e.g. one or few hours, up to one or few days) in order to provide the quantity of cells necessary to achieve an optimal effect. Doses for administration may be variable (e.g. between 10² to 10¹⁰ or more cells per each CBMP formulation), as it can be determined by team at the medical facility according to the specific disease (e.g. chronic, acute, viral, inherited, etc.) and/or to factors individual to each patient (e.g. weight, age, size of tissue damage, amount of time since the disease occurred, past or future treatments based on immunosuppressive or therapeutic drugs, genetic background, etc.).

As described above, MCF Facility has structural and technical features that are distinct from those described previously in the field of production of biological for pharmaceutical use and that make MCF Facility at the same time flexible and functional for delivering CBMP formulations at (or nearby) medical sites that are differently located and equipped, increasing the safety and reproducibility of all operations. Thus, MCF Facility is a suitable environment for both producing and temporarily storing CBMP formulations at specific temperature conditions, while being of a very limited size, easy to move in different locations, and requiring almost no preparation at the medical site where CBMP formulations will be finally administered.

An example of the use of MCF Facility involves the formulation of a bulk CBMP preparation (being primary, positively selected bone marrow cells, genetically modified or reprogrammed embryonic or induced stem cells, or in vitro expanded adult stem or progenitor cells) that have been manufactured in GMP conditions as a batch (i.e. a defined quantity of CBMP that have produced in the series of processes so that it could be expected to be homogeneous) in a plant within one country (e.g. Belgium) and then distributed and formulated at a medical site in the same country and/or in another one (e.g. France, Great Britain, or Italy). Once that bulk CBMP preparation has been cryopreserved into appropriate bags, these bags are transferred in the Working Area 1.3 of MCF Facility that, following the official approval of local regulatory competent authorities, can be moved across one or more of such distant locations. MCF Facility maintains the bulk CBMP preparation, as well as all materials that are needed for the CBMP formulation at the medical site (e.g. excipients, media, chemicals) within the expected range of temperature during the transportation and, once arrived at destination (e.g. an indoor or an outdoor parking area of the medical site, providing at least access to electric power and possibly also to water, liquid nitrogen, and/or communication networks), during any of the required operations, and later on when preparing a further CBMP formulation in the same medical facility, moving MCF Facility to another medical site, or going back to the original bulk CBMP manufacturing facility.

MCF Facility allows starting to produce CBMP formulations within First Working Area quickly (e.g. within less than two hours from the arrival) which is compliant to compliant with ISO 14644-1 Class 7 standard. One or more bags of bulk CBMP preparation (each containing 10⁷, 10⁸, 10⁹ or more cells, at a concentration of 10⁴, 10⁵, 10⁶ or more cells per ml) are used according to established procedures and technologies (e.g. based on Sepax technology) for handling CBMPs while connecting collection bags, syringes, needles, tubes, and vials and adding media buffers, growth factors, or other drugs according to GMP specifications. A basic procedure may include cell washing (using a closed system), formulation (using a mixing, closed system with disposable bags filled with appropriate medium), and vial/bags/syringe filling (using any Restricted Area Barrier System). This process yields one or more aseptic and temperature-controlled CBMP formulations ready to be administered, each containing cells that are prepared in GMP conditions, and at concentration and/or in a total amount that can be (depending on the specific treatment or patient) similar, superior, or inferior to the values as initially determined in a single bag of the bulk CBMP preparation.

In-process Quality control operations for monitoring (and, if necessary adjusting) the process and ensuring that CBMP formulation conforms to its specification are performed in Second Working Area (1.2; FIGS. 3A, 3B and 3E), using retention samples of CBMP Formulation that are transferred from Working Area 1.1.A, by verifying if the quality and concentration standards are met, e.g. by evaluating cell concentration and/or viability (e.g. >50% after 6 hours from formulation), absence of biological contaminants (e.g. as determined by dye- or antibody-based assays), presence of specific surface antigens on the cells in CBMP formulation (e.g. by using flow cytometry, ELISA, or other antibody-based assays), antigen-specific interactions or activation, and/or the concentration of chemicals and cryopreservative that may be still present after washing CBMPs (e.g. DMSO below 5000 ppm), with the scope of checking if GMP conditions were actually maintained in the environment of MCF Facility before CBMP formulation is actually administered to a patient.

Once that these parameters are evaluated, the CBMP formulation that was temporarily stored in Working Area 1.3.B (e.g. up to 8 hours) can be transferred from MCF Facility to the local medical team which is in charge of administering said formulation to a patient that was previously prepared for allogeneic, matched or unmatched cell-based therapies (e.g. by treating with immunosuppressors, by administering anaesthetics, and/or implanting a catheter).

In this manner, a CBMP that is registered or in a clinical phase of validation (i.e. for establishing documented evidence that provides a high degree of assurance that a CBMP will consistently and safely provide the intended therapeutic effects), can be successfully formulated, validated as being conform to specifications, and distributed sequentially in one or more medical sites using an alternative, more efficient route than making use only of GMP-compliant in-plant manufacturing and formulation procedure that is represented by a single MCF Facility. Thus, it is evident how MCF Facility provides companies and medical institutions with a mobile and easily adapted structure for distributing CBMPs, in a much more reliable and efficient manner than known standardized laboratory or pilot production facilities for biological products, for establishing allogeneic, somatic cell-based therapies. For example, MCF Facility has been successfully used for distributing CBMP formulations containing preparations of Adult-Derived Human Liver Mesenchymal-Like Cells (Najimi M et al., 2007) in medical sites where they were administered to patients, such sites being up to 1000 kilometres far from the bulk CBMP manufacturing facility.

It will be apparent to those of skill in the art that, in light of the present disclosure, variations may be applied to the structure, the equipment, and the assembly of MCF Facility and in the steps or in the sequence of steps of the methods and uses described herein without departing from the scope of the disclosure and without undue experimentation.

The terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated. As a consequence, all modifications and alterations will occur to others upon reading and understanding the previous description. In particular, dimensions, materials, and other parameters, given in the above description may vary depending on the needs of the application.

Reference numerals in the claims do not limit their protective scope.

Use of the verbs “to comprise”, “to include”, “to be composed of”, or any other variant, as well as their respective conjugations, does not exclude the presence of elements other than those stated.

Use of the article “a”, “an” or “the” preceding an element does not exclude the presence of a plurality of such elements.

Although certain examples have been described herein, the scope of coverage of this patent is not limited thereto. On the contrary, this patent covers all methods, apparatus, and articles of manufacture fairly falling within the scope of the appended claims either literally or under the doctrine of equivalents.

1. A facility for formulating a Cell-Based Medicinal Product comprising:
a first unit that is a working space to allow operators to at least one of store, count, test, sample, formulate, or package cell preparation and produce a Cell-Based Medicinal Product formulation according to Good Manufacturing Practices;
a second unit adjacent to and communicatively coupled the first unit to allow operators to access to the first unit, to store equipment and materials, to prepare themselves before and after accessing the first unit, and to bring Cell-Based Medicinal Product preparations, the equipment, and the materials in and out of the first unit;
a third unit adjacent to the first unit, the third unit to house equipment for exchanging of at least one of electric power, air, or data with one or more of the first unit, the second unit or a fourth unit; and
the fourth unit that includes a vehicle to transport a unitary structure comprising the first unit, the second unit, and the third unit.

2. The facility of claim 1, wherein the first unit comprises:
a first working area equipped with a vertical laminar air flow coupled to the third unit with one or more instruments for at least one of manipulating, sampling, formulating, or packaging cell preparations under sterile conditions;
a second working area equipped with one or more instruments for at least one of counting or testing cell preparations; and
a third working area equipped for storing at least one of cell preparations or equipment to be used within the first unit.

3. The facility of claim 1 further comprising a fifth unit positioned between, and communicatively coupled by means of doors with the first unit and the second unit only.

4. The facility of claim 1, wherein the first unit comprises walls are made of composite panels comprising:
a water- and air-tight inner surface;
an intermediate layer of a thermally insulating material; and
a shock-resistant, water- and air-tight external surface.

5. The facility of claim 1, wherein the third unit comprises an HVAC system to exchange air and regulate temperature in the first unit.

6. The facility of claim 5, wherein the HVAC system is to exchange air and regulate temperature in the second unit.

7. The facility of claim 5 further comprising a fifth unit, wherein the HVAC system is to exchange air and regulate temperature in the fifth unit.

8. The facility of claim 2 further comprising one or more instruments for at least one of acquiring, monitoring, storing, elaborating, or transmitting data related to at least one of the preparation of Cell-Based Medicinal Product formulations, manipulations made in the facility, and a status of the first unit, the second unit, the third unit, the fourth unit, the first working area, the second working area, the third working area, or equipment in the facility.

9. The facility of claim 1, wherein the facility is to allow operating according to ISO standards that are applicable to at least one of production, formulation, administration, or medical use of cell-based products.

10. The facility of claim 1, wherein the first unit is equipped to allow operators to formulate and validate a Cell-Based Medicinal Product comprising at least one of stem or progenitor cells, primary cells, or cells that have been genetically modified.

11. The facility of claim 1, wherein the fourth unit comprises a truck.

12. A method of providing formulations of a Cell-Based Medicinal Product in a site where said formulations are administered to a patient, comprising:
building a unitary structure comprising equipment and working spaces that allow operators to at least one of store, formulate, or validate Cell-Based Medicinal Products according to Good Manufacturing Practices, and assembling said structure with a vehicle for establishing a facility;
providing bulk Cell-Based Medicinal Products that are prepared according to Good Manufacturing Practices;
storing said bulk Cell-Based Medicinal Product preparations within the facility; and
moving the facility to a site where said bulk Cell-Based Medicinal Product preparations are used for producing and validating formulations of Cell-Based Medicinal Product within said facility, and where said formulations are administered to a patient.
